(19) United States
(12) Patent Application Publication
(10) Pub. No.: US 2006/0184101 A1
(43) Pub. Date: Aug. 17, 2006

(54) Microjet devices and methods for drug delivery

(76) Inventors: Ravi Srinivasan, Mountain View, CA (US); Ruben Rathnasingham, San Mateo, CA (US)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(21) Appl. No.: 11/367,202

(22) Filed: Mar. 3, 2006

Related U.S. Application Data
(63) Continuation-in-part of application No. 10/829,888, filed on Apr. 21, 2004.
(60) Provisional application No. 60/463,905, filed on Apr. 21, 2003.
(60) Provisional application No. 60/483,604, filed on Jun. 30, 2003.
(60) Provisional application No. 60/492,342, filed on Aug. 5, 2003.
(60) Provisional application No. 60/658,389, filed on Mar. 4, 2005.

(51) Int. Cl. A61M 5/30

(52) U.S. Cl. 604/68

(57) ABSTRACT

A fluid delivery system includes a reservoir, a delivery actuator, and at least one delivery nozzle of a microjet having an exit orifice with a diameter between about 1 μm and about 500 μm. The delivery actuator may be configured to deliver a quantity of fluid contained in the reservoir into tissue of an individual through the nozzle or nozzles at a pre-determined velocity, and to desired depths. The quantity of fluid may contain one or more therapeutic agents, such as medications, drugs, bio-reactive agents, etc. The delivery actuator may also be configured to repeatedly deliver a quantity of the fluid contained in the reservoir through the at least one delivery nozzle at pre-determined intervals.

### CROSS-REFERENCE TO RELATED APPLICATIONS

This application is a continuation-in-part of U.S. application Ser. No. 10/829,888 filed Apr. 21, 2004, which claims priority to U.S. Provisional Application Nos. 60/463,905, filed Apr. 21, 2003; 60/483,604, filed Jun. 30, 2003; and 60/492,342 filed Aug. 5, 2003; each of which is incorporated herein by reference in their entirety. This application also claims priority to U.S. Provisional Application No. 60/658,389 filed Mar. 4, 2005 entitled “Microjet Devices for Drug Delivery,” which is incorporated herein by reference in its entirety.

### BACKGROUND OF THE INVENTION

### The Field of the Invention

Generally the present invention relates to the field of delivering therapeutic agents, such as drugs. More particularly, the present invention provides devices and methods for the delivery of therapeutic agents using microjets.

One method of drug delivery is transdermal drug delivery. Transdermal drug delivery is the delivery of the drug substance directly across the skin barrier. Transdermal drug delivery has been in existence for roughly two decades. Transdermal delivery has many advantages over other drug delivery methods, including avoiding first pass metabolism and the ability to maintain consistent systemic dosage levels avoiding the peaks and troughs experienced with other drug delivery methods. Furthermore, transdermal drug delivery is an extremely convenient dosage vehicle for the patient and tends to achieve high levels of patient compliance.

The main barrier to diffusion of pharmaceuticals across the skin is the outermost layer of the skin, the stratum corneum. The stratum corneum consists of densely packed keratinocytes (flat dead cells filled with keratin fibers) surrounded by highly ordered lipid bilayers, creating an effective barrier to permeability. Directly beneath the stratum corneum is the viable epidermis. The viable epidermis is rich in cells of the immune system, and therefore a target for drug delivery for therapies that are directed to or involve the immune system. Beneath the epidermis is the dermis. The dermis has a rich network of blood capillaries and, therefore, is an attractive target for systemic drug delivery since drugs presented to the capillary network rapidly enter the circulatory system and are systemically delivered throughout the body.

Various methods for enhancing transdermal drug delivery across the stratum corneum have been devised including utilizing enhancing agents or stimulants such as chemical, voltage charge, ultrasonic waves, thermal treatments, microneedles, and laser assist techniques. For example, see U.S. Pat. No's. 6,352,506 and 6,216,033. However, the development and broad acceptance of these methods has been hampered by skin irritation, incompatibility with the drug formulations, and the complexity and expense of the devices themselves. Furthermore, these techniques may not offer the capability of time-dependent dosage delivery, which is crucial to many therapeutics, including insulin.

One mechanism of drug delivery across the stratum corneum is the use of needless injections or high-speed jet injectors. High-speed jet injectors have been utilized as hypodermic syringe replacements for many years. For example, see U.S. Pat. Nos. 2,380,534, 4,596,556, 5,520,639, 5,630,796, 5,993,412 and 6,913,605. Jet injectors move the solution to be injected at a high rate of speed and eject the solution as a jet, penetrating the stratum corneum and depositing the solution into the dermis and subcutaneous tissues.

While traditional high-speed jets are capable of transporting drugs across the stratum corneum, a drawback of this mechanism is that they deliver a large quantity of the composition being delivered in a one-time bolus jet injection. As a result, some of the drug is often forced back out of the penetration pore. Moreover, the one-time delivery fails to maintain a sustained systemic drug concentration at therapeutic levels. Still further, due to the large quantity of drug delivered at one-time, patients often experience skin irritation, pain, swelling, and other undesirable effects similar to injections with hypodermic syringes.

U.S. Patent Publication No. 2004/0260234 discloses the use of high-speed microjets created by driving a volume of fluid, about 1 pl to about 800 nl, via a single nozzle with a diameter of about 1 μm to 500 μm or an array of such nozzles. The speed of fluid expelled from the jets can be very high, with velocities greater than 30 m/s but typically about 100 m/s. In contrast inkjet printers generate fluid velocities of about 5 m/s. Repetitive delivery by the high-speed jets can be realized in several ways including, spring actuation, high-pressure gas, phase change leading to rapid pressure increase, electromagnetic means, such as by using a solenoid, piezoelectric means, etc.

Other methods of drug delivery include catheters and intravenous injections. These methods are particularly invasive and do not easily deliver precisely targeted amounts of a therapeutic agent to a specific area. For example, it may be desirous to deposit a small amount of medication directly into the heart muscle, without the medication moving throughout the body and potentially causing unintended side-effects for other organs and tissue. Current catheter and intravenous methods for drug delivery do not allow the required precision, which requires injection of drugs in quantities far higher than actually necessary.

Less-invasive and more precise techniques of drug delivery by using microjets for sustained transdermal and intravenous delivery to a specific, desired location of a composition at consistent therapeutic levels to a patient are highly desirable.

### BRIEF SUMMARY OF THE INVENTION

Some aspects of the present invention may include a fluid delivery system having a reservoir, a delivery actuator, and at least one delivery nozzle of a microjet having an exit orifice with a diameter between about 1 μm and about 500 μm. The delivery actuator may be configured to deliver a quantity of fluid contained in the reservoir through the nozzle or nozzles at a pre-determined velocity. The quantity of fluid may contain one or more therapeutic agents, such as medications, drugs, bio-reactive agents, etc. The delivery actuator may also be configured to repeatedly deliver a quantity of the fluid contained in the reservoir through the at least one delivery nozzle at pre-determined intervals, at intervals determined by a treatment provider or the individual using the system, and at pre-determined velocities.

In some aspects, the system may be configured to deliver a quantity of fluid at a velocity such that the quantity of fluid disrupts and passes into and/or through the stratum corneum of an individual into the epidermal layer, dermal layer, or below, of an individual.

In another aspects, the system may include at least one nozzle is located on a distal end of a catheter and/or endoscope. In such aspects the system may be configured to deliver a quantity of fluid directly into the bloodstream, or to some other portion of an individual proximate to the nozzle. For example, the system may be configured to deliver the quantity of fluid through a vascular wall or into tissue adjacent to a vascular wall of an individual, or into other tissues, body fluids, regions, etc. reached with an endoscope, including into the spinal column.

In some other aspects the delivery of a quantity of fluid may be based on a signal from a sensor. The sensor may be a biosensor such as a pressure sensor, density sensor, chemical sensor, or an electrical sensor. The sensor may be located inside of the individual to be treated, or may be monitoring or attached to machinery monitoring conditions of the individual. Similarly, the microjet delivery device may be located externally, as a transdermal delivery device, or internally, to deliver therapeutic agents to a desired location.

In other aspects, the system may be configured such that the pre-determined velocity delivers the quantity of fluid onto the stratum corneum of an individual without disrupting the stratum corneum. The fluid may be delivered onto the stratum corneum of an individual through an intermediate member, such as an absorbent material, patch, etc.

In some aspects the system may be configured to deliver a quantity of fluid into the nasal cavity of an individual. The fluid may be delivered through tissues in the nasal cavity of the individual, either by depositing the fluid onto the nasal membranes, or by delivering the fluid into the nasal cavity tissues, or below, by penetrating the tissue with the fluid. The fluid may also be delivered onto tissues in the nasal cavity by misting the quantity of fluid through the at least one delivery nozzle. Similarly, the system may be configured to deliver fluid through the mouth and/or throat tissues of an individual, by misting, depositing, or penetration. The delivery of fluid may also be configured to be inhaled and absorbed in the lungs of the individual when, for example, it is misted into the nasal cavity, mouth, and/or throat.

In some aspects, the delivery system may include a plurality of nozzles. In some such aspects, a at least a first portion of the delivery nozzles may be configured as high speed nozzles, and at least a second portion of the plurality of nozzles may be configured as low speed nozzles. The high speed nozzles may be configured to disrupt the stratum corneum and create pores, and the low speed nozzles may be configured to deliver a quantity of fluid through the created pores, either directly or through an intermediate member such as an absorbent patch. In some configurations, the high speed nozzles may be reconfigured as low speed nozzles, such that the high speed function and the low speed function are accomplished by the same nozzles.

These and other aspects of the present invention will become more fully apparent from the following description and claims.

### BRIEF DESCRIPTION OF THE DRAWINGS

For a better understanding of the nature and objects of the invention, reference should be made to the following detailed description, read in conjunction with the accompanying drawings, in which:

FIG. 1 is schematic view of an embodiment of a microjet device;

FIG. 2 is a schematic view of an embodiment of a microjet device;

FIG. 3 is a schematic view of an embodiment of a microjet device having a catheter and/or endoscope portion;

FIG. 4 is a schematic view of an embodiment of a microjet device;

FIG. 5 is a schematic view of an embodiment of a microjet device and including an intermediate delivery member;

FIG. 6 is a schematic view of an embodiment of a microjet device;

FIG. 7 is a schematic view of an embodiment of a microjet device and including an intermediate delivery member;

FIG. 8 is a schematic view of an embodiment of a microjet device and including an intermediate delivery member and an iontophoresis system;

FIG. 9 is a schematic view of an embodiment of a microjet device having a catheter and/or endoscope portion;

FIG. 10 is a schematic view of an embodiment of a microjet device having a catheter and/or endoscope portion;

FIG. 11 is a schematic view of an embodiment of a microjet device having a catheter and/or endoscope portion;

FIG. 12 is a schematic view of an embodiment of a microjet device having a catheter and/or endoscope portion;

FIG. 13 is a schematic view of an embodiment of a microjet device having a sensor;

FIG. 14 is a schematic view of an embodiment of a microjet device;

FIG. 15 is a schematic view of an embodiment of a microjet device;

FIG. 16a is a schematic view of an embodiment of a microjet device;

FIG. 16b is a schematic view of an embodiment of a microjet device;

FIG. 17 is a schematic view of an embodiment of a microjet device;

FIG. 18a is a schematic view of an embodiment of a microjet device;

FIG. 18b is a schematic view of an embodiment of a microjet device;

FIG. 19a is a schematic view of an embodiment of a microjet device;

FIG. 19b is a schematic view of an embodiment of a microjet device;

FIG. 20a is a schematic view of an embodiment of a microjet device; and

FIG. 20b is a schematic view of an embodiment of a microjet device;

### DETAILED DESCRIPTION OF EXEMPLARY EMBODIMENTS OF THE INVENTION

Reference will now be made in detail to the preferred embodiments of the invention, examples of which are illustrated in the accompanying drawings. While the invention will be described in conjunction with the preferred embodiments, it will be understood that they are not intended to limit the invention to those embodiments. On the contrary, the invention is intended to cover alternatives, modifications, and equivalents, which may be included within the spirit and scope of the invention as defined by the appended claims. For ease of reference, feature numbering is consistent throughout the various embodiments discussed below and presented in the Figures.

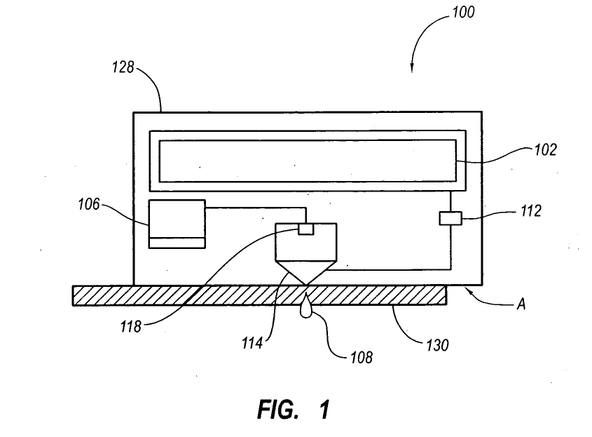

Referring now to a microjet device 100 as shown in FIG. 1, a fluid reservoir 102 is in fluid communication with a microjet 114 that is controlled by a controller 106, which may be a microprocessor, or any other suitable controller. Controller 106 is programmable to activate an actuator 118 to propel a quantity of fluid 108 from microjet 114 towards a biological barrier, such as the stratum corneum 130 of an individual. Microjet 114, as shown throughout the disclosure, includes an exit nozzle with an opening of between about 1 μm and about 500 μm. This small opening of the microjet 114 may minimize pain and tissue damage to an individual receiving treatment via microjet device 100.

Furthermore, the microjet device 100 is capable of repeatable activation. For the sake of clarity, repeatable activation is defined to mean multiple, sequential activation without the need to remove, recharge, or otherwise replenish the device between activation cycles and deactivation cycles. For example, a particular drug administration regime may require delivery of a particular quantity of the drug on each hour for five days. In this example, the microjet device would activate an actuator 118 to inject as many micro injections as needed to deliver the prescribed quantity of drug at the first hour. Upon completion of a first hour's administration, the device would wait until the next hour, and then administer the prescribed quantity of drug a second time. The device would then continue in this manner for the entire five day period.

Moreover, according to some embodiments, controller 106 may be a simple electronic component or control unit that generates a signal according to predetermined or preprogrammed timing to activate the microjet 114 to propel quantity of fluid 108 from reservoir 102. The signal may also determine the velocity of fluid 108 expelled from microjet 114, depending on the desired delivery regimen. The velocity may be controlled various ways such as by adjusting the size of the microjet nozzle, controlling the force applied by the actuator, adjusting the size of the actuator, etc. Similarly, several factors may determine the speed of delivery such as the viscosity of fluid 108, the length of travel between actuator 118 and microjet 114, the elasticity of materials used in constructing various components of microjet device 100, etc. Such factors may be taken into account in determining the velocity of the microjet discharge.

Generally, the velocity of fluid 108 may be between 0.1 m/s and 150 m/s, depending on the application, as discussed more fully below. The timing of the signal can be sequential, but is not limited to sequential timing. The signal may also control valve 112 to determine the quantity of fluid 108 or duration of the delivery cycle. Actuator 118, may be driven by one or more of several different mechanisms including piezoelectric, solenoid, vaporization pressure, etc., as described in U.S. Patent Publication No. 2004/0260234.

Reservoir 102, as shown in FIG. 1, is configured to house a substance to be ejected from microjet 104. Fluid 108 may contain one or more therapeutic agents, such as medications, drugs, bio-reactive agents, etc. Typically, fluid 108 is in a liquid form at the time of injection and may be a drug composition, saline solution, emulsion of drug in fluid media, suspension of drug in fluid media, drug coated liposomes in fluid media, drug or drug coated particulates in fluid media, etc.

According to some embodiments, as exemplified in FIG. 2, controller 106 may control an array of microjets 114. The array of microjets 114 may deliver a larger quantity of a substance 108 across a larger surface area than the single microjet 114 of FIG. 1. The array of microjets 114 may also deliver multiple substances and/or deliver substances in a pattern to optimize administration of a particular substance. Similarly, groups of microjets 114, or each microjet 114 may be separately controlled to deliver fluids at different velocities, quantities, or a plurality of fluids.

For simplicity and clarity the following description will primarily describe in detail the components of the single microjet device 100, as shown in FIG. 1. Reference will be made to the array embodiment, such as that shown in FIG. 2, however, it should be appreciated that the description of the components is equally applicable to each embodiment and not limited to an embodiment utilizing a single microjet 114.

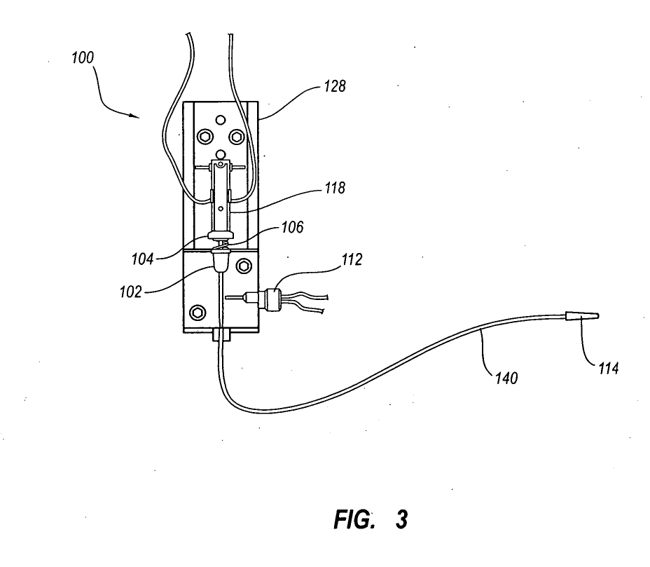

In some embodiments, as shown in FIG. 3, the microjet 114 may be located at the distal end of an endoscope and/or catheter 140. The endoscope and/or catheter 140 allows for manipulation and location of microjet nozzle 114 at to desired target location. In such embodiments, microjet device 100 may include a housing 128, an actuator 118, a reservoir 102, catheter and/or endoscope 140, and may be remotely controlled and/or powered. Microjet device 100 may also include a piston 104 and a spring 106.

In one example, actuator 118 may be a piezo-electric actuator that drives piston 104 when activated. Piston 104 may then reduce the volume of reservoir 102, causing microjet device 100 to discharge a quantity of fluid 108 contained in reservoir 102 through the nozzle of microjet 114. In one embodiment, spring 106 may bias actuator 118 and piston 104 together. When actuator 118 is actuated and drives piston 104, piston 104 may continue to travel away from actuator 118 due to the momentum of piston 104. Spring 106 may then return piston 106 to its original position in contact with actuator 118. In another embodiment (not pictured), actuator 118 may be bonded to piston 104 such that actuator 118 and piston 104 travel simultaneously during activation of actuator 118.

In some embodiments, the catheter and/or endoscope tubing outer diameter may be any conventional size, and preferably varies from about 1 mm to about 1 cm, most preferably from 1 mm to 3 mm. The catheter tubing inner diameter may be any conventional size, and preferably varies from about 0.5 mm to about 9 mm, most preferably from 1 mm to 5 mm. The speed of the microjet delivery for catheter and/or endoscope-based delivery may be from about 1 m/s to about 50 m/s (in air), and may preferably be from about 1 m/s to about 10 m/s (in air).

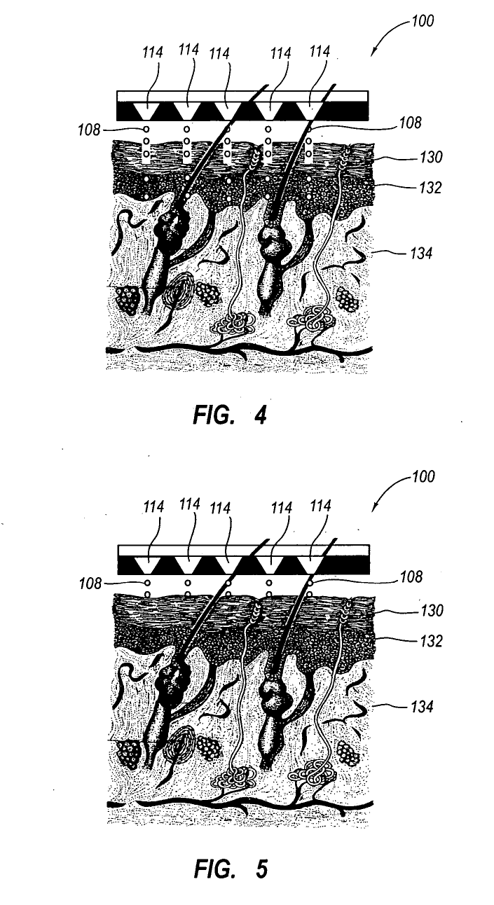

In some embodiments, as shown in FIG. 4, microjet 114 may discharge fluid 108 with a velocity sufficient to disrupt the stratum corneum 130. Adjustments to the velocity may allow fluid 108 to deliver therapeutic agents to the stratum corneum 130, the viable epidermis 132, the dermis 134, or to tissues below the dermal layer. The speed of the microjet delivery across stratum corneum 130 may be from about 1 m/s to about 150 m/s, depending on the desired depth. In some embodiments, the speed may be preferably between 10 m/s and 100 m/s for delivery into the viable epidermis 132 and/or the dermis 134. In these embodiments, through control of the velocity of the discharge, therapeutic agents may be delivered with precision to the layer where the therapeutic agent will be most effective.

In some embodiments, microjets 114 may disrupt the stratum corneum with high velocity delivery of fluid 108, and then deliver additional therapeutic agents with a low velocity delivery through the pores created by the high velocity delivery. In some of these embodiments, microjets 114 may be configured such that some of microjets 114 are configured for high velocity delivery, while other microjets 114 are configured for low velocity delivery. In such embodiments, the speed of the microjet 114 configured for low velocity may be from about 0.1 m/s to about 5 m/s, and preferably from about 0.1 m/s to about 0.5 m/s (in air). In other embodiments, microjets 114 may be configured to first disrupt the stratum corneum 130 with a high velocity delivery, the velocity of microjets 114 may then be adjusted to low velocity delivery for subsequent delivery through the pores created by the high velocity delivery.

Similarly, as shown in FIG. 5, the velocity of microjets 114 may be adjusted such that fluid 108 is delivered as droplets onto the skin surface but does not disrupt the stratum corneum 130. In such embodiments, the speed of microjet 114 may be from about 0.1 m/s to about 5 m/s, and preferably from about 0.1 m/s to about 0.5 m/s (in air). Therapeutic agents in fluid 108 diffuse from the top of the skin surface across the stratum corneum barrier for systemic delivery.

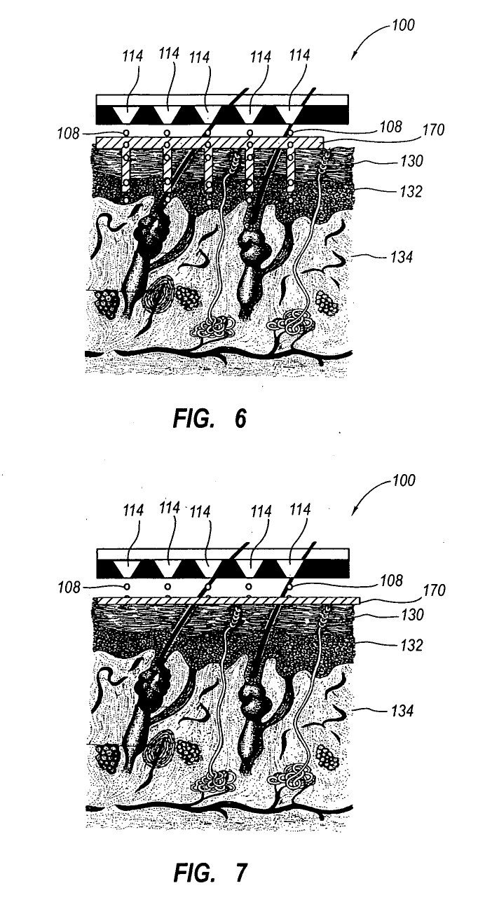

FIGS. 6, 7, and 8 show embodiments where an intermediate member 170 is placed on the stratum corneum 130 with subsequent delivery from intermediate member 170. As shown in FIGS. 6 and 8, some embodiments may include intermediate member 170 protruding into stratum corneum 130 after the stratum corneum 130 is disrupted by microjets 114. FIG. 7 shows an embodiment where intermediate member 170 provides fluid 108 to an undisrupted stratum corneum. FIG. 8 shows subsequent drug delivery achieved using an ionotophoresis system 172.

Intermediate member 170 may be pre-medicated, or continuously or periodically loaded with fluid 108 from microjet system 100. In such embodiments, the speed of microjet 114 may be from about 0.1 m/s to about 5 m/s, and preferably from about 0.1 m/s to about 0.5 m/s (in air). Intermediate member 170 may be an absorbent pad placed against the skin surface with a subsequent diffusion of a therapeutic agent from the pad into the body. Intermediate member 170 may be a porous polymeric material that is flexible to conform to the body contours. Porex Inc. and Micropore Inc. manufacturer materials suitable for use as intermediate member 170.

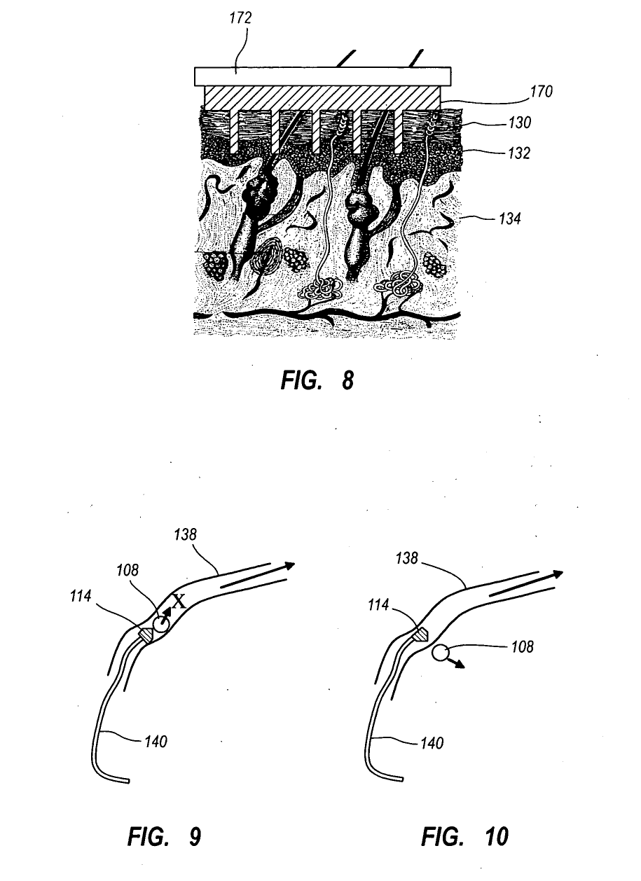

FIGS. 9-12 show embodiments of the microjet device 100 that may include catheter and/or endoscope 140. Catheter and/or endoscope 140 may be used to deliver therapeutic agents strategically and precisely to portions of the body in need of a particular therapeutic agent. Examples of therapeutic agents suitable for precise placement may include anti-clotting agents, drugs for arthroscopic plaque removal, drugs that prevent restinosis after an angiography, anti-cancer therapies, anesthetics, etc. location indicated by the X in FIG. 9. Microjet 114 may deliver pulses of fluid 108 into the vasculature, including arteries and veins. The speed of the microjet for vascular delivery may be from about 1 m/s to about 50 m/s (in air), preferably from about 5 m/s to about 30 m/s, and most preferably from about 10 m/s to about 20 m/s. As shown in FIG. 10, microjet 114 may also be used to deliver drugs across vascular wall 138 into adjoining tissues. The energy of the microjet pulse may be tuned to ensure that microjet 114 creates a micropore on the vascular wall 138 at the delivery site.

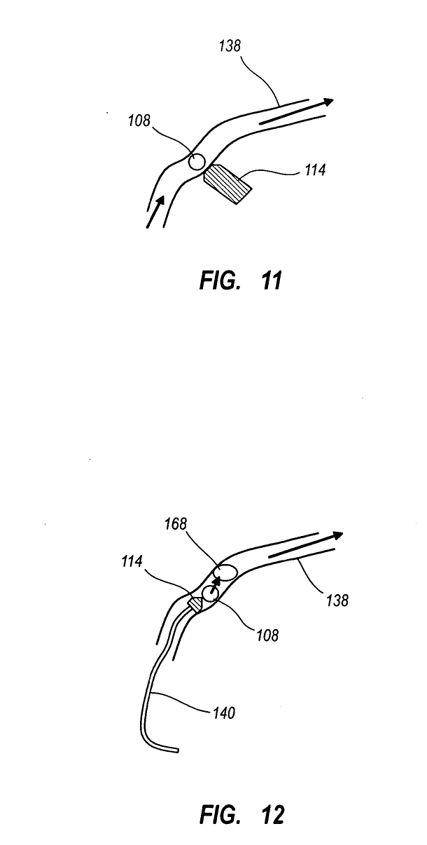

Similarly, as shown in FIG. 11, microjet 114 may also delivery fluid 108 into a blood vessel across the vascular wall 138 from outside of the vessel. The velocity of microjet 114 may be adjusted to enter the artery or vein but does not damage the vascular wall 138 on the far side of delivery site. In embodiments where microjet 114 delivers fluid 108 across a vascular wall 138, microjet 114 may be adjusted such that microjet 114 may be placed in contact with vascular wall 138 or adjacent but at a distance away from vascular wall 138. The distance between the nozzle of microjet 114 and vascular wall 138 may vary from about 1 to 20 mm.

One example of a method for using the catheter and/or endoscope microjet device 110, is shown in FIG. 12. In the example, microjet 114 is placed in proximity to plaque or clot 168 in blood vessel 138. Microjet 114 directs fluid 108 including a therapeutic agent effective to reduce or destroy plaque or clot 168 directly to plaque or clot 168, thereby achieving the desired result of removing or reducing plaque or clot 168, using a minimal amount of therapeutic agent, and causing minimal damage to other body tissues and organs.

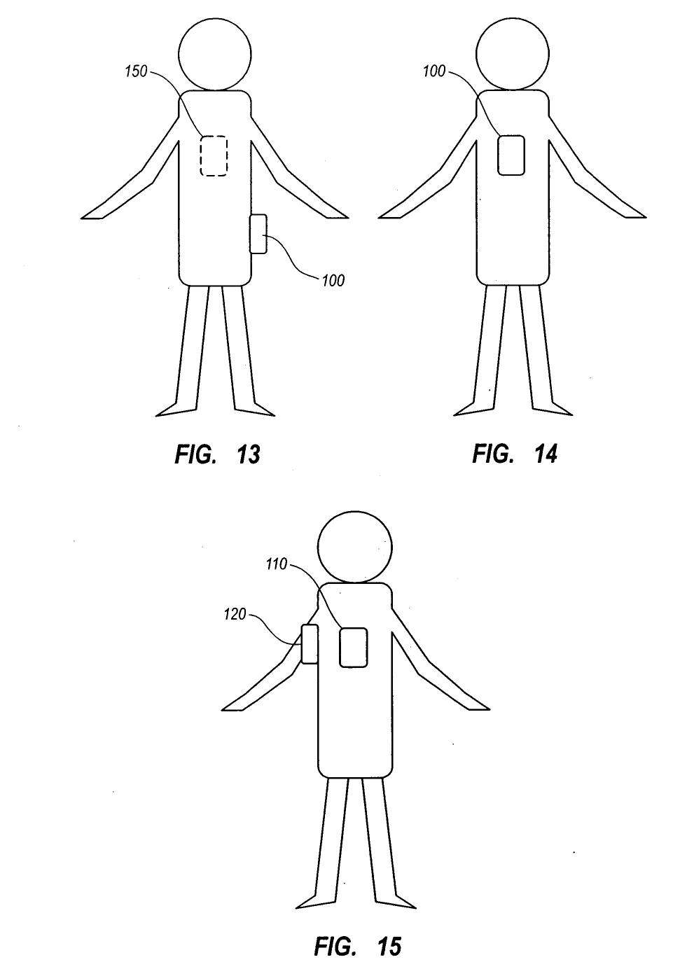

In other embodiments, as shown in FIG. 13, microjet system 100 may deliver therapeutic agents transdermally in response to a signal from an implantable device or sensor 150. Implantable device 150 as shown in FIG. 13 is located in the thoracic region of the body for illustrative purposes but may be located in any region of the body, including, for example, in the skin under the stratum corneum. The communication between implanted device 150 and microjet system 100 may be via wireless means or by means of a conducting wire.

One example may include an implantable defibrillator or pacemaker as implanted device or sensor 150 and an externally located microjet system 100 for transdermal delivery. In such an example, if a cardiac event occurs, the implantable defibrillator or pacemaker 150 detects the event and relays the signal to the microjet system 100, which delivers appropriate therapeutic agents. Some examples of therapeutic agents useful in this example may include blood-modifying agents such as heparin and streptokinase, inotropic agents such as dobutamine, dopamine, digoxin and milrinone, etc.

Implanted device or sensor 150 may be any one of or a combination of an implantable electrode that detects the onset of a central nervous system attack such as seizures, an electrode pair or electrode array implanted in the brain, in the spinal cord, or on other organs that records neural readings, chemical sensors such as cell-based biosensors, glucose sensors, protein-based biosensors, sensors based on absorbance, emittance or fluorescence of electromagnetic waves, sensors measuring electrical property changes such as but not limited to resistance, capacitance, voltage, and inductance, sensors measuring mass uptake such as but not limited to resonant frequency and resonance damping, miniature pressure sensors or pressure sensors to measure body fluid pressure at a particular location in the body, including blood pressure, intra-cranial pressure in the brain or in the spinal cord, and intra-ocular pressure in the eye, etc.

Similarly, as shown in FIG. 14, microjet system 100 may be implanted in an individual. Microjet system 100 may be used for dosing and metering of therapeutic agents including small molecules and macromolecules. Microjets 114 may also be used for delivering drugs across biological barriers and into organs. For example, implanted microjet device 100 could be used to deliver medications into the heart, stomach, liver, lungs, eyes, pancreas and such organs. Implanted microjet device 100 may also be used for site-specific drug delivery such as localized drug delivery into cancerous tissue, such as chemotherapy agents to cancerous tissue which may reduce or eliminate the need for systemic chemotherapy agent delivery, as currently practiced, reducing the undesirable side effects of the chemotherapy agents on healthy tissue.

As shown in FIG. 15, recharging implanted microjet system 100 may be accomplished using an external device that generates radio-frequency energy. The radio-frequency energy may then be used to charge the battery of implanted microjet system 100.

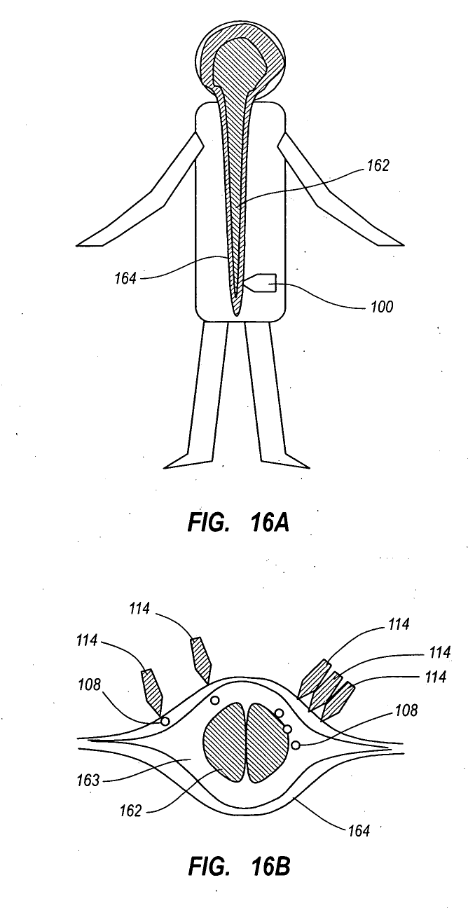

Embodiments shown in FIGS. 16a and 16b may use microjet system 100 to deliver therapeutic agents directly into the central nervous system (CNS). Some therapeutic agents that may be delivered using this approach may include those that target the CNS but cannot pass through the blood-brain barrier. Some examples of such therapeutic agents may include dopamine, oncology drugs and psychiatric drugs. Microjets 114 may be used to deliver fluid 108 including therapeutic agents to various targets in the CNS as shown in FIG. 16b. For example, fluid 108 may be delivered to the spinal or cranial meninges 138 for the treatment of local inflammation from meningitis. For another example, therapeutic agents may be delivered into the intra-thecal space 166 for localized therapy, for example anesthesia, or transported through the entire CNS by the circulating cerebro-spinal fluid (CSF) 163. Similarly, a microjet or an array of microjets may be used at specific spatial locations on the spinal or cranial meninges to address more targeted therapies. This technique may be used to target specific motor or sensory neural tracts on spinal cord 162.

The velocity of fluid 108 from microjet 114 can be adjusted to determine the injection depth. For example, very high velocities, from about 20 m/s to about 100 m/s, may be used to deliver therapeutic agents into the CSF 163, or even into the spinal cord 162, while moderate velocities, from about 1 m/s to 30 m/s, may be used to deliver therapeutic agents into the meninges 164 but not into the CSF 163. When the nozzle of microjet 114 is placed adjacent to the dura (biological barrier covering the brain and spinal cord) and in contact with the dura, the momentum of fluid 108 may serve to deform the vascular wall and create a micropore in the dura. Microjets 114 may also be operated adjacent but at a distance away from the dura at a distance from about 1 mm to about 20 mm.

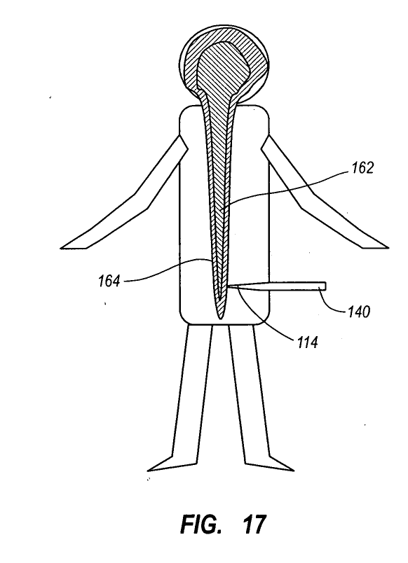

FIG. 17 shows another embodiment that may use microjets 114 to deliver therapeutic agents across the blood-brain barrier. Microjet 114 may be placed inside of or at the distal end of a needle or a catheter 140 that is inserted percutaneously. The needle may be made from a rigid polymer or metal while the catheter could be fabricated from flexible polymeric materials. The outer diameter of the needle or catheter may be from about 100 μm to 5 mm, preferably from about 500 μm to 1 mm. The nozzle of microjet 114 may be placed adjacent to the meninges without penetrating it. When actuated, the high-speed jet penetrates the meninges to deliver drugs to the intra-thecal space 166 that circulates and delivers the therapeutic agent throughout the central nervous system. The required velocity of fluid 108 from microjet 114 to penetrate the dura and deliver a target injection depth is the same as discuss with respect to FIGS. 15a and 15b.

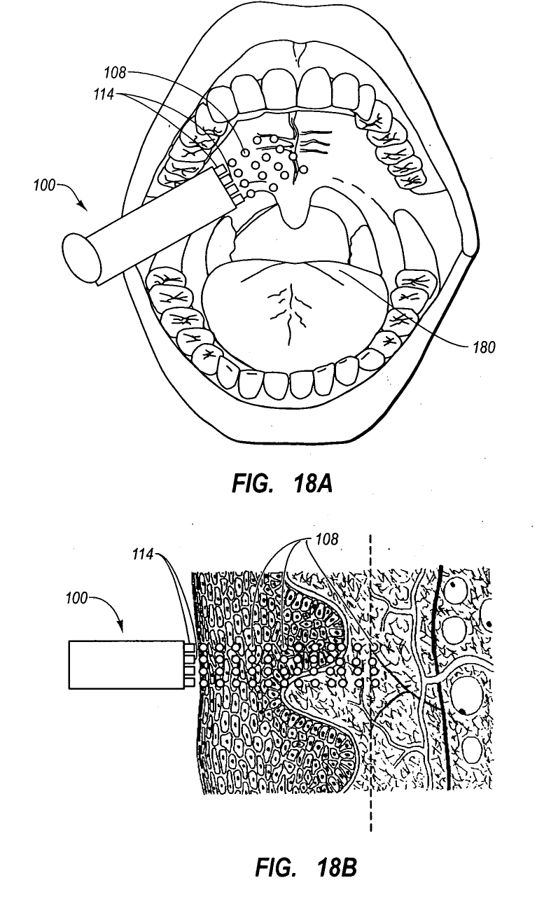

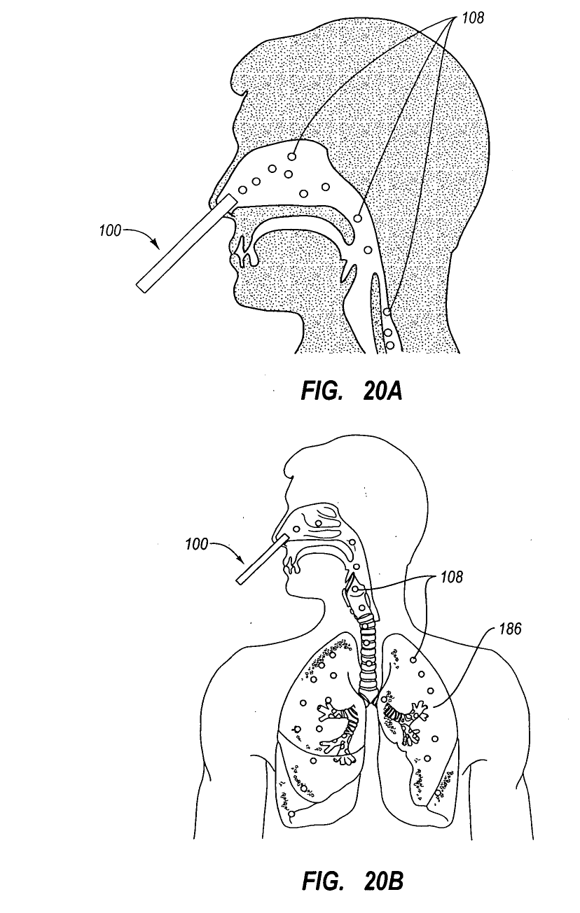

FIGS. 18a-20b show embodiments of microjet system 100 delivery to transmucosal and pulmonary tissues via the oral cavity 180 and nasal cavity. As shown in FIGS. 18a, 18b, and 20a, the nozzle(s) of the microjets 114 may be placed against the mucosal lining in the mouth or the nose, and high speed fluid 108 from the microjet device 100 may penetrate the epithelial barrier and deposit therapeutic agent at a pre-determined fixed depth just underneath epithelial barrier 182. Oral-transmucosal and nasal-transmucosal drug delivery may be an attractive route for delivering both small and large molecules since the epithelium of the mucosa is soft in comparison with the stratum corneum of the skin. Furthermore, the mucosal lining is bereft of langerhans cells, reducing the risk of an immune response due to drug delivery.

While oral and nasal transmucosal drug delivery using high-speed microjets has been discussed in detail, this method of drug delivery may be broadly applicable to transmucosal drug delivery in general including and not limited to rectal-transmucosal and vaginal-transmucosal drug delivery. Fluid media based microjets (liquids, solids suspended in liquids) as well as solids and powder based microjets delivered at high speeds may be used to overcome the mucosal barrier.

Another embodiment of the microjet device based transmucosal therapeutic agent delivery may deposit therapeutic agent microdroplets on the outer layers of the epithelium of the mucosa but not damage or penetrate the epithelium. In this embodiment microjet device 100 may used for precise volume control and dosing. The route of administration includes but is not limited to oral-transmucosal, nasal-transmucosal, rectal-transmucosal and vaginal-transmucosal.

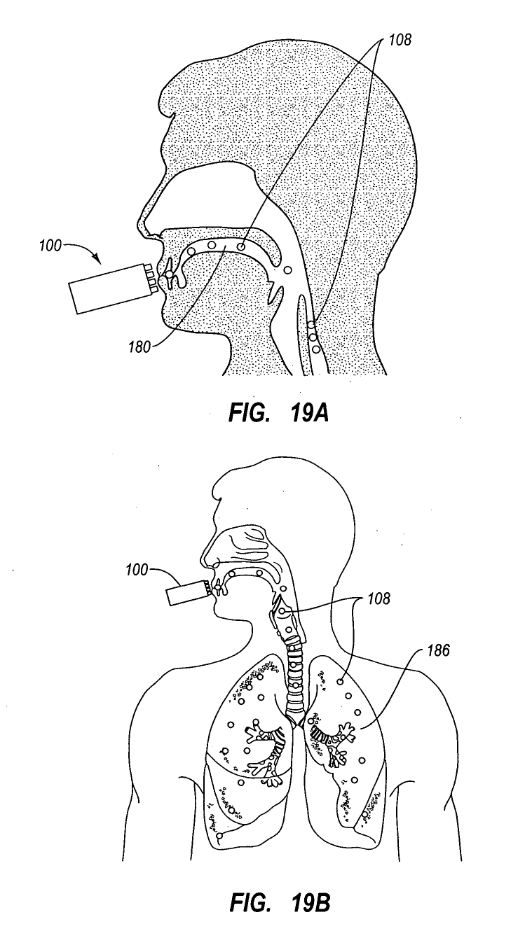

As shown in FIGS. 19a-20b, microjet device 100 may also be used to generate aerosols of drugs that can be inhaled via the mouth 180, as shown in FIGS. 19a and 19b, or via the nose, as shown in FIGS. 20a and 20b, for delivery into the blood stream via the alveoli of the lungs 186.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A fluid delivery system, comprising:
a reservoir;
a delivery actuator;
at least one delivery nozzle having an exit orifice with a diameter between about 1 μm and about 500 μm; and
wherein the delivery actuator is configured to deliver a predetermined quantity of a fluid contained in the reservoir through the at least one delivery nozzle at a pre-determined velocity.

2. The fluid delivery system of claim 1, wherein the system is configured to deliver the quantity of fluid at a velocity such that the quantity of fluid disrupts and passes into and/or through the stratum corneum of an individual.

3. The fluid delivery system of claim 2, wherein the system is configured to deliver the quantity of fluid into one of the epidermal layer, the dermal layer, and subdermal tissue of an individual.

4. The fluid delivery system of claim 1, wherein the at least one nozzle is located on a distal end of a catheter.

5. The fluid delivery system of claim 4, wherein the system is configured to deliver the quantity of fluid into the bloodstream of an individual.

6. The fluid delivery system of claim 4, wherein the system is configured to deliver the quantity of fluid through a vascular wall of an individual.

7. The fluid delivery system of claim 1, wherein the delivery of a quantity of fluid is based on a signal from a sensor.

8. The fluid delivery system of claim 7, wherein the sensor is a biosensor selected from one or more of a pressure sensor, density sensor, chemical sensor, and an electrical sensor, and wherein the sensor configured to be located at least one of internally and externally of the individual.

9. The fluid delivery system of claim 1, wherein the system is configured to deliver the quantity of fluid onto the stratum corneum of an individual.

10. The fluid delivery system of claim 9, wherein the quantity of fluid is delivered onto the stratum corneum of an individual through an intermediate member.

11. The fluid delivery system of claim 1, wherein the system is configured to deliver the quantity of fluid into one or more of the mouth, the throat, and the nasal cavity the nasal cavity of an individual.

12. The fluid delivery system of claim 11, wherein the quantity of fluid is delivered through tissues in one or more of the mouth, the throat, and the nasal cavity of the individual.

13. The fluid delivery system of claim 11, wherein the quantity of fluid is delivered onto tissues in one or more of the mouth, the throat, the lungs, and the nasal cavity by misting the quantity of fluid through the at least one delivery nozzle.

14. The fluid delivery system of claim 11, wherein the delivery of the quantity of fluid is configured to be inhaled and absorbed in the lungs of the individual.

15. The fluid delivery system of claim 1, wherein the at least one delivery nozzle is a plurality of nozzles, and wherein at least a first portion of the delivery nozzles are high velocity nozzles, and a second portion of the plurality of nozzles are low velocity nozzles.

16. The fluid delivery system of claim 15, wherein the high velocity nozzles are configured to create pores in the stratum corneum of an individual by disrupting the stratum corneum, and the low velocity nozzles are configured to deliver the quantity of fluid through the created pores.

17. The fluid delivery system of claim 1, wherein the fluid includes at least one therapeutic agent.

18. The fluid delivery system of claim 1, wherein the delivery actuator is configured to repeatedly deliver a quantity of the fluid contained in the reservoir through the at least one delivery nozzle at pre-determined intervals.

19. The fluid delivery system of claim 1, wherein the system is configured to deliver the quantity of fluid at a velocity such that the quantity of fluid disrupts and passes into and/or through the dura around the spinal column and/or the brain of an individual.

20. The fluid delivery system of claim 19, wherein the system is configured to deliver the quantity of fluid into the meninges of an individual.

21. The fluid delivery system of claim 19, wherein the system is configured to deliver the quantity of fluid into the cerebro-spinal fluid of an individual.

22. A method of fluid delivery, comprising:
providing a fluid delivery device, wherein the fluid delivery device includes at least one microjet having a nozzle with a diameter between about 1 μm and about 500 μm;
determining a desired penetration depth in a target region of an individual, wherein the penetration depth is less than 3 cm;
locating the fluid delivery device in contact with or adjacent to the target region;
controlling delivery of a fluid through the nozzle of the at least one microjet at a velocity required to deliver the fluid to about the determined penetration depth.

23. The method of claim 22, wherein the target region includes on one of skin, mucosal tissue, vascular tissue, central nervous system, and internal organs of an individual.

24. The method of claim 22, wherein the fluid delivery device is implanted in the individual.

25. The method of claim 22, wherein the controlled delivery is based on a signal from a sensor.

26. The method of claim 25, wherein the sensor is a biosensor selected from one or more of a pressure sensor, density sensor, chemical sensor, and an electrical sensor, and wherein the sensor configured to be located at least one of internally and externally of the individual.
